(19) United States
(12) Patent Application Publication
(10) Pub. No.: US 2019/0255007 A1
(43) Pub. Date: Aug. 22, 2019

(54) METHOD OF TREATING PAIN WITH CHLOROGENIC ACID

(71) Applicant: Vidya Herbs, Inc., Fullerton, CA (US)

(72) Inventor: Kodimule Shyam Prasad, Bangalore (IN)

(73) Assignee: Vidya Herbs, Inc., Fullerton, CA (US)

(21) Appl. No.: 16/261,543

(22) Filed: Jan. 29, 2019

Related U.S. Application Data
(60) Provisional application No. 62/623,634, filed on Jan. 30, 2018.
(60) Provisional application No. 62/701,804, filed on Jul. 22, 2018.

(51) Int. Cl. A61K 31/216; A61P 25/00; A61K 9/00; A61K 36/74

(52) U.S. Cl. CPC A61K 31/216; A61K 36/74; A61K 9/0053; A61P 25/00

(57) ABSTRACT

A composition of chlorogenic acid and a method of its use in treating pain are provided. The composition can be made from botanical materials, including green coffee bean. The composition and can be used in treating somatic, visceral and neuropathic pain, including acute and chronic pain.

### CROSS-REFERENCE TO RELATED APPLICATIONS

This application claims priority to Provisional Application No. 62/623,634 filed Jan. 30, 2018 and Provisional Application No. 62/701,804 filed Jul. 22, 2018. The entire contents of these applications are incorporated herein by reference for all purposes.

### FIELD OF THE INVENTION

The invention generally relates to a nutraceutical composition for the treatment of pain.

### BACKGROUND

Pain is the most common manifestation of many disorders afflicting millions of people worldwide. Pain is not easily or satisfactorily defined and therefore is often interpreted as a suffering that results from the perception of painful stimuli. Pain is a common symptom and can indicate that something is wrong in the body and may give a clue to the nature of disease. Hence, pain is a specific sensation with its own peripheral and central mechanisms independent of other senses. While pain itself is not a disease, it is by far the most common medical complaint. Pain is mainly considered a defense mechanism created when a tissue is damaged causing a reaction to remove a pain stimulant (Baradaran et al. 2013). In severe conditions, pain impairs social functioning and reduces quality of life (Bonica, 1979). It is usually perceived as an indication of ill health and most diseases have a component of pain.

Drugs which mitigate pain sensitivity or remove pain are called painkillers, or analgesics. Even though there are effective allopathic medications used to alleviate the manifestation of pain, many such medications have side effects.

Vanilloid receptors are recognized as a molecular integrator of painful stimuli ranging from noxious heat to endovanilloids in inflammation. TrpV1 (transient receptor potential cation channel subfamily V member 1) and TrpV4 are two vanilloid receptors implicated in propagating painful stimuli.

TrpV1 is a nonselective cation channel that may be activated by a wide variety of exogenous and endogenous physical and chemical stimuli. Activators of TrpV1 include: temperature greater than 43° C. (109° F.); acidic conditions; capsaicin (the irritating compound in hot chili peppers); and isothiocyanate (the pungent compound in mustard and wasabi) (Everaerts et al. 2011). The activation of TrpV1 leads to a painful, burning sensation. TrpV1 is involved in the transmission and modulation of pain, as well as the integration of diverse painful stimuli (Cui et al. 2006; Huang et al. 2002).

Initial approaches targeting TrpV1 utilized agonists for the treatment of pain. Capsaicin is the prototypical TrpV1 agonist that is found in many topical formulations (Anand & Bley, 2011). Agonist treatment for pain is related to the initial excitation of sensory neurons followed by a refractory state of desensitization where the neuron becomes unresponsive to TrpV1 agonists and other inflammatory mediators. Repeated or high dose agonist application produces a reversible ablation of the nerve fiber that further reduces sensitivity to cutaneous stimuli such as tactile, heat, mechanical and cold stimuli (Nolano et al. 1999). However, because this later approach essentially produces a neurolytic lesion of TrpV1-associated epidermal nerve fibers, the physiology for pain alleviation is mechanistically different from antagonist approaches.

Pharmacological blockade of TrpV1 represents a new strategy in pain relief. TrpV1 antagonists are expected to prevent pain by silencing receptors generated rather than stopping the propagation of pain as most traditional pain killers do. This hypothesis has already been tested in the clinic by administering small molecule TrpV1 antagonists (e.g. GlaxoSmithKline SB-705498) for migraine and dental pain. TrpV1 antagonists have shown efficacy in reducing pain perception from inflammatory and neuropathic pain models in rats (Jhaveri et al. 2005). The discovery of selective TrpV1 antagonists has provided additional support for the role of TrpV1 channels in inflammatory pain conditions. Pre-clinically, TrpV1 antagonists are effective at blocking thermal hypersensivity to numerous inflammogens (e.g. carrageenan, complete Freund's adjuvant, and CFA), without modulating associated inflammatory responses, as measured by edema. Initial clinical results indicate that TrpV1 antagonists decrease thermal pain perception in normal subjects and elevate core body temperature (Round et al. 2011; Rowbotham et al. 2011).

Modulation of TrpV1 signaling has been shown to have effects other than the regulation of pain. According to a study led by Andrew Dillin of the University of California Berkeley, mice lacking TrpV1 live longer and have a more youthful metabolism than age-matched wild-type mice. This effect is believed to involve a novel pathway centered on calcitonin gene-related peptide (CGRP). Like ablating TrpV1, blocking CGRP receptors in aged wild-type mice restored their metabolism to a more youthful state. This study highlights the emerging overlap between pain and metabolism. Located on the peripheral endings and cell bodies of dorsal root ganglion (DRG) neurons, TrpV1 channels can be activated by scalding heat and the irritant capsaicin. TrpV1 activation leads to CGRP release by DRG neurons of the trigeminal nucleus, which contributes to migraine pain. Moreover, TrpV1-positive DRG neurons innervating the pancreas regulate insulin release through CGRP (Gram et al., 2007) increasing the potential for obesity.

### SUMMARY OF THE INVENTION

The inventor discovered a unique formulation of chlorogenic acids and successfully investigated its potential for the management of pain.

It is therefore an object to provide a method of treating pain comprising administering to a patient in need thereof a composition comprising chlorogenic acid.

In some aspects, the composition comprises 3-caffeoylquinic acid (3-CQA), 5-caffeoylquinic acid (5-CQA), 4-caffeoylquinic acid (4-CQA), 5-feruloylquinic acid (5-FQA), 3,4-dicaffeoylquinic acid (3,4-diCQA), 3,5-dicaffeoylquinic acid (3,5-diCQA), and 4,5-dicaffeoylquinic acid (4,5-diCQA).

In other aspects, the composition comprises 3-CQA, 5-CQA, 4-CQA, 5-FQA, 3,4-diCQA, 3,5-diCQA, and 4,5-diCQA in a ratio of about 2.6:9.4:3.4:1.0:1.2:1.0:1.6, by weight.

In still other aspects, the composition comprises 3-CQA, 5-CQA, 4-CQA, 5-FQA, 3,4-diCQA, 3,5-diCQA, and 4,5-diCQA in a ratio of about 2.7:10.2:3.7:1.0:1.3:1.0:1.7, by weight.

In other aspects, the composition comprises about 13 w/w % 3-CQA, about 47 w/w % 5-CQA, about 17 w/w % 4-CQA, about 5 w/w % 5-FQA, about 6 w/w % 3,4-diCQA, about 5 w/w % 3,5-diCQA, and about 8 w/w % 4,5-diCQA.

In some aspects, the composition comprises a mixture of chlorogenic acids, wherein the mixture has about 13% 3-CQA, about 47% 5-CQA, about 17% 4-CQA, about 5% 5-FQA, about 6% 3,4-diCQA, about 5% 3,5-diCQA, and about 8% 4,5-diCQA.

In some aspects, the composition comprises 12.5 w/w % 3-CQA, 46.9 w/w % 5-CQA, 17.2 w/w % 4-CQA, 4.6 w/w % 5-FQA, 6.2 w/w % 3,4-diCQA, 4.6 w/w % 3,5-diCQA, and 8.0 w/w % 4,5-diCQA.

In some aspects, the composition comprises a mixture of chlorogenic acids, wherein the mixture has 12.5% 3-CQA, 46.9% 5-CQA, 17.2% 4-CQA, 4.6% 5-FQA, 6.2% 3,4-diCQA, 4.6% 3,5-diCQA, and 8.0% 4,5-diCQA.

In other aspects, the administered composition comprises 3,5-diCQA. The composition can comprise about 5 w/w % 3,5-diCQA or about 4.6 w/w % 3,5-diCQA.

In still other aspects, the administered composition consists essentially of 3,5-diCQA.

### BRIEF DESCRIPTION OF THE DRAWINGS

FIG. 1 shows an LC chromatogram of an embodiment of the inventive composition.

FIG. 2 shows an MS chromatogram of an embodiment of the inventive composition.

FIG. 3 shows a graphical representation of the binding of 3,5-diCQA with the calmodulin binding site of TrpV1.

FIGS. 4A and 4B show the RMSD values of the 3,5-diCQA acid TrpV1 protein-ligand complex (FIG. 4A) and the molecule alone (FIG. 4B).

FIG. 5 shows graphical representations of the molecular interaction of the following chlorogenic acid isomers with the vanilloid binding site of TrpV1: (A) 3-caffeoylquinic acid; (B) 4-caffeoylquinic acid; (C) 5-caffeoylquinic acid; (D) 3,4-Dicaffeoylquinic acid; (E) 3,5-Dicaffeoylquinic acid; and (F) 4,5-Dicaffeoylquinic acid.

FIG. 6 shows a graphical representation of ATP bound to the ankyrin repeat domain of TrpV1.

FIG. 7 shows graphical representations of the molecular interaction of the following chlorogenic acid isomers with the ATP-calmodulin binding site of TrpV1: (A) 3-caffeoylquinic acid; (B) 4-caffeoylquinic acid; (C) 5-caffeoylquinic acid; (D) 3,4-Dicaffeoylquinic acid; (E) 3,5-Dicaffeoylquinic acid; and (F) 4,5-Dicaffeoylquinic acid.

FIG. 8 shows a graphical representation of ATP bound to the ankyrin repeat domain of TrpV4.

FIG. 9 shows graphical representations of the molecular interaction of the following chlorogenic acid isomers with the ATP-calmodulin binding site of TrpV4: (A) 3-caffeoylquinic acid; (B) 4-caffeoylquinic acid; (C) 5-caffeoylquinic acid; (D) 3,4-Dicaffeoylquinic acid; (E) 3,5-Dicaffeoylquinic acid; and (F) 4,5-Dicaffeoylquinic acid.

### DEFINITIONS

As used herein, the term “about” means the quantity, level, value, number, frequency, percentage, dimension, size, amount, weight or length that is referenced, or that varies (plus or minus) by as much as 30%, 25%, 20%, 15%, 10%, 9%, 8%, 7%, 6%, 5%, 4%, 3%, 2% or 1% to the referenced quantity, level, value, number, frequency, percentage, dimension, size, amount, weight or length.

As used herein, the phrases “effective amount,” “effective dose,” and “therapeutically effective amount” refer to that amount of an agent that is sufficient to ameliorate a disorder, disease or condition, or the symptoms of a disorder, disease or condition. For example, an effective amount may be an amount of a composition that is sufficient to prevent or alleviate the sensation of pain.

As used herein, the term “reduce” refers to any measurable decrease in a parameter relative to control conditions.

The terms “subject,” “patient,” “individual” are used interchangeably herein to refer to, except where indicated, mammals such as humans and non-human primates, as well as livestock and companion and laboratory research animals. The terms can refer to an individual that has been diagnosed with pain, is experiencing pain, is currently following a therapeutic regimen for pain, or is at risk of developing pain.

As used herein, the terms “therapy,” “treating,” “treat,” and “treatment” refer to preventing, arresting, inhibiting the progression of, reducing the severity of the sensation of pain.

Throughout this specification, unless the context requires otherwise, the words “comprise,” “comprises,” and “comprising” will be understood to imply the inclusion of a stated step or element or group of steps or elements, but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. The phrase “consisting essentially of” means including any elements listed after the phrase and excluding any other elements that interfere with or contribute to the activity or action of the listed elements. Thus, the phrase “consisting essentially of” indicates that the listed elements are required or mandatory, but that other elements are optional if they do not materially affect the activity or action of the listed elements.

### DETAILED DESCRIPTION

The invention generally relates to a method of treating pain. More particularly, the invention relates to a use of a chlorogenic acid composition in the treatment of pain.

In some non-limiting embodiments, the invention provides a method of treating pain comprising administering to a patient in need thereof an effective amount of a composition comprising at least one chlorogenic acid. The composition can comprise 3-caffeoylquinic acid (3-CQA), 5-caffeoylquinic acid (5-CQA), 4-caffeoylquinic acid (4-CQA), 5-feruloylquinic acids (5-FQA), 3,4-dicaffeoylquinic acids (3,4-diCQA), 3,5-dicaffeoylquinic acids (3,5-diCQA), 4,5-dicaffeoylquinic acids (4,5-diCQA), or combinations thereof. The composition can comprise a mixture of chlorogenic acids selected from the group consisting of 3-CQA, 5-CQA, 4-CQA, 5-FQA, 3,4-diCQA, 3,5-diCQA, 4,5-diCQA, and combinations thereof. In one non-limiting aspect of the invention, the composition comprises 3,5-diCQA. In a further non-limiting embodiment, the composition consists essentially of 3,5-diCQA. It will be understood that in some embodiments, the chlorogenic acid isomers disclosed herein include, without limitation, analogues, derivatives and modifications of the disclosed chlorogenic acid isomers.

Some aspects of the invention concern the relative amounts of the chlorogenic acids that are present in the composition. The composition can comprise about 13 w/w % 3-CQA, about 47 w/w % 5-CQA, about 17 w/w % 4-CQA, about 5% w/w % 5-FQA, about 6 w/w % 3,4-diCQA, about 5 w/w % 3,5-diCQA, about 8 w/w % 4,5 di CQA, or combinations thereof. The composition can comprise chlorogenic acids selected from the group consisting of about 13 w/w % 3-CQA, about 47 w/w % 5-CQA, about 17 w/w % 4-CQA, about 5 w/w % 5-FQA, about 6 w/w % 3,4-diCQA, about 5 w/w % 3,5-diCQA, about 8 w/w % 4,5 di CQA, and combinations thereof. The composition can comprise 12.5 w/w % 3-CQA, 46.9 w/w % 5-CQA, 17.2 w/w % 4-CQA, 4.6 w/w % 5-FQA, 6.2 w/w % 3,4-diCQA, 4.6 w/w % 3,5-diCQA, 8.0 w/w % 4,5 di CQA, or combinations thereof. The composition can comprise chlorogenic acids selected from the group consisting of 12.5 w/w % 3-CQA, 46.9 w/w % 5-CQA, 17.2 w/w % 4-CQA, 4.6 w/w % 5-FQA, 6.2 w/w % 3,4-diCQA, 4.6 w/w % 3,5-diCQA, 8.0 w/w % 4,5 di CQA, and combinations thereof. In one non-limiting embodiment, the composition consists essentially of about 5 w/w % 3,5-diCQA. In a further non-limiting embodiment, the composition consists essentially of 4.6 w/w % 3,5-diCQA.

In at least one embodiment, the chlorogenic acid isomers of the composition are present in a weight-to-weight ratio. The ratio can be a ratio that corresponds to any of the percentages disclosed herein. By way of one non-limiting example, a composition comprising 13 w/w % 3-CQA, about 47 w/w % 5-CQA, about 17 w/w % 4-CQA, about 5% w/w % 5-FQA, about 6 w/w % 3,4-diCQA, about 5 w/w % 3,5-diCQA, and about 8 w/w % 4,5-diCQA can be expressed as a ratio of about 2.6:9.4:3.4:1.0:1.2:1.0:1.6. Similarly, a composition comprising 12.5 w/w % 3-CQA, 46.9 w/w % 5-CQA, 17.2 w/w % 4-CQA, 4.6 w/w % 5-FQA, 6.2 w/w % 3,4-diCQA, 4.6 w/w % 3,5-diCQA, and 8.0 w/w % 4,5-diCQA can be expressed as a ratio of 2.7:10.2:3.7:1.0:1.3:1.0:1.7. The composition can comprise a mixture of chlorogenic acids, wherein the mixture comprises 3-CQA, 5-CQA, 4-CQA, 5-FQA, 3,4-diCQA, 3,5-diCQA, and 4,5-diCQA a ratio of about 2.7:10.2:3.7:1.0:1.3:1.0:1.7. The composition can comprise a mixture of chlorogenic acids, wherein the mixture consists of, or essentially of, 3-CQA, 5-CQA, 4-CQA, 5-FQA, 3,4-diCQA, 3,5-diCQA, and 4,5-diCQA a ratio of about 2.7:10.2:3.7:1.0:1.3:1.0:1.7.

In some embodiments, the composition comprises a mixture of chlorogenic acids, wherein the mixture comprises about 13% 3-CQA, about 47% 5-CQA, about 17% 4-CQA, about 5% 5-FQA, about 6% 3,4-diCQA, about 5% 3,5-diCQA, and about 8% 4,5-diCQA. The composition can comprise a mixture of chlorogenic acids, wherein the mixture consists of about 13% 3-CQA, about 47% 5-CQA, about 17% 4-CQA, about 5% 5-FQA, about 6% 3,4-diCQA, about 5% 3,5-diCQA, and about 8% 4,5-diCQA. The composition can comprise a mixture of chlorogenic acids, wherein the mixture consists of 12.5% 3-CQA, 46.9% 5-CQA, 17.2% 4-CQA, 4.6% 5-FQA, 6.2% 3,4-diCQA, 4.6% 3,5-diCQA, and 8.0% 4,5-diCQA.

In some aspects of the invention, the composition comprises at least one excipient that is in contact with one or more chlorogenic acids as disclosed herein. The excipient can be selected on the basis of compatibility with the chlorogenic acids and the properties of the desired dosage form. Excipients include, but are not limited to, binders, fillers, flow aids/glidents, disintegrants, lubricants, stabilizers, surfactants, and the like. A summary of suitable excipients include, but are not limited to, those disclosed in Remington: The Science and Practice of Pharmacy, 19^{th} Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999). The entire contents of these and other documents referred to in this specification are incorporated herein by reference for all purposes.

In at least one embodiment, the composition can comprise controlled, sustained, or extended release formulations known collectively as “modified release” formulations. Examples of non-limiting modified release and delivery systems include, but are not limited to, those described in the following U.S. patents, the entire disclosures of which are incorporated herein by reference for all purposes: U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566. Dosage forms for the composition can be used to provide modified release of one or more active ingredients using, for example, hydropropylmethyl cellulose, other polymer matrices, gels, permeable membranes, osmotic systems, multilayer coatings, microparticles, liposomes, microspheres, and combinations thereof.

At least one aspect of the invention concerns the form of the composition. The composition can be in the form selected from the group consisting of a powder, liquid, pill, tablet, pellet, capsule, thin film, solution, spray, syrup, linctus, lozenge, pastille, chewing gum, paste, vapor, suspension, emulsion, ointment, cream, lotion, liniment, gel, drop, topical patch, buccal patch, bead, gummy, gel, sol, injection, and combinations thereof. The composition can be formulated for oral administration. The composition can be in contact with at least one vitamin, mineral, extract, amino acid, protein, carbohydrate, lipid, fatty acid, food, beverage, nutritional or dietary supplement, excipient, pharmaceutically acceptable carrier, bulking agent, binding agent, caffeine, flavoring, sweetener, preservative, or combinations thereof. In at least one aspect of the invention, the composition is provided and/or manufactured in bulk. The composition can be provided in bulk for the manufacture of foods, nutritional supplements, nutraceuticals, dietary supplements and/or food supplements for the treatment of pain. Bulk quantities of the composition can be packaged, stored and/or distributed in drums, bags, boxes, containers and the like. Such containers can be configured to prevent or inhibit the oxidation of the active ingredients of the composition.

At least one aspect of the invention concerns a method of making the composition. The composition can be made by combining one or more of the chlorogenic acids disclosed herein. For example, the composition can be made by combining one or more of the disclosed chlorogenic acids in isolated form. In some non-limiting embodiments, the composition is made from an extract of a botanical material. The composition can be an extract of a botanical material. The botanical material can comprise any botanical material capable of providing the chlorogenic acid formulations disclosed herein. Suitable botanical materials include, but are not limited to, coffee beans, sunflower seeds, tea, blueberries, honeysuckle, guayusa leaf, bamboo (Phyllostachys edulis), peaches, prunes, heather (colluna vulgaris), chinese parsley, potatoes, tomatoes, apples, tobacco, eggplant, lonicera flowers (jinyinhua), eucommia bark, gardenia fruit, chrysanthemum flower, crataegus fruit, artemisia leaves, epimedium leaves, artichoke leaves, burdock root, dandelion root, echinacea root, flaxseeds, strawberries, pineapple fruit, peanuts, wheat or combinations thereof. In one non-limiting embodiment, the composition comprises an extract of green coffee beans, dried coffee beans, roasted coffee beans, or combinations thereof. Examples of suitable coffee beans include, but are not limited to, Cofea Arabica, Cofea Robusta, and the like. In one non-limiting embodiment of the invention, the composition comprises an extract of green coffee beans. In some aspects, the composition is an extract of coffee cherry, coffee cherry mucilage, or a combination thereof.

The composition can be made from the botanical substance using any suitable process for collecting chlorogenic acids, such processes including, without limitation, solvent extraction, extrusion, or a combination thereof. Suitable solvents for obtaining extracts for obtaining the composition include, but are not limited to, aqueous solvents, alcohol-based solvents, supercritical fluids, polar organic solvents (such as acetone and methylethyl ketone), or combinations thereof. Non-limiting examples of alcohol-based solvents include, but are not limited to, ethanol, isopropyl alcohol, methanol, and combinations thereof. The supercritical fluid can be, but is not necessarily limited to, carbon dioxide.

In some aspects, the invention provides a method of treating pain in a patient in need thereof. The method can be practiced by administering to the patient the composition disclosed herein. The composition can be administered in an effective amount. The pain can be somatic pain, visceral pain, neuropathic pain, or combinations thereof. The pain can be acute pain or chronic pain. The pain can be nociceptive pain, inflammatory pain, pathological pain, or combinations thereof. Pain treatable by the invention includes, without limitation, migraine, headaches (including tension-type and cluster headaches), trigeminal neuralgia, herpetic neuralgia, general neuralgias, postherpetic neuralgia, cramping (including menstrual cramping), psychogenic pain, allodynia, carpal tunnel syndrome pain, fibromyalgia pain, arthritis pain (including rheumatoid arthritis and osteoarthritis pain), pain due to sunburn, peripheral neuropathy pain, diabetic neuropathy pain, radicular pain, sciatica, back pain, head and neck pain, hyperalgesia, spontaneous pain, phantom pain, idiopathic pain, pain in the skin and musculoskeletal tissues, itching, pain from insect stings and bites, dysesthesia (including thermal and cold dysesthesia), severe or intractable pain, breakthrough pain, persistent pain, tendinitis pain, postsurgical pain, cancer pain, dental pain, muscle aches, pain due to injury including sports and occupational injuries (including burns, abrasions, fractures, sprains, strains, tears, contusions, cuts, and the like). In at least one aspect, treating pain means reducing, arresting or preventing the sensing of pain by a patient. In at least one embodiment, the invention provides a bandage, wound dressing, or the like which is in contact with (such as infused or saturated with) a composition disclosed herein.

Some aspects of the invention concern the administration of the composition. The composition can be administered systemically and/or locally. The composition can be administered locally, such as the site of pain, for example. Suitable administration routes for the composition include, but are not limited to, auricular, buccal, conjunctival, cutaneous, dental, endocervical, endosinusal, endotracheal, enteral, epidural, extra-amniotic, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal dental, intracoronary, intracorporus cavernosum, intradermal, intradiscal, intraductal, intraduodenal, intradural, intraepidermal, intraesophageal, intragastric, intravaginal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, oropharyngeal, parentera, percutaneous, periarticular, peridural, perineural, periodontal, rectal, inhalation, retrobulbar, soft tissue, subarachnoid, subconjunctival, subcutaneous, sublingual, submucosal, topical, transdermal, transmucosal, transplacental, transtracheal, transtympanic, ureteral, urethral, vaginal, or combinations thereof. The composition can be administered by irrigation, drip, infusion, or topically by a dressing, patch, or bandage that is in contact with the composition.

Some aspects of the invention concern the dosage of the composition. The composition can be administered at a dose of between about 5 mg/day and about 500 mg/day. The composition can be administered at a dose of between about 20 mg/day and about 1 mg/day. The composition can be administered at a dose of about 20 mg/day, about 21 mg/day, about 22 mg/day, about 23 mg/day, about 24 mg/day, about 25 mg/day, about 26 mg/day, about 27 mg/day, about 27 mg/day, about 28 mg/day, about 29 mg/day, about 30 mg/day, about 31 mg/day, about 32 mg/day, about 33 mg/day, about 34 mg/day, about 35 mg/day, about 40 mg/day, about 45 mg/day, about 50 mg/day, about 100 mg/day, about 150 mg/day, about 200 mg/day, about 250 mg/day, about 300 mg/day, about 350 mg/day, about 400 mg/day, about 450 mg/day, or about 500 mg/day, as well as any dosage intervening these specifically disclosed amounts. The composition can be administered at a dosage of between about 400 mg/day and about 500 mg/day, between about 300 mg/day and about 400 mg/day, between about 200 mg/day and about 300 mg/day, between about 100 mg/day and about 200 mg/day, between about 100 mg/day and about 200 mg/day, or about 20 mg/day and about 100 mg/day. It is contemplated that the composition can be administered at any dosage that intervenes the dosages called out in this specification.

In some aspects, the composition is administered according to the body weight of the patient. The composition can be administered between about 5 mg/kg b.w. and about 500 mg/kg b.w. The composition can be administered at about 5 mg/kg b.w., about 10 mg/kg b.w., about 20 mg/kg b.w., about 30 mg/kg b.w., about 40 mg/kg b.w., about 50 mg/kg b.w., about 60 mg/kg b.w., about 70 mg/kg b.w., about 80 mg/kg b.w., about 100 mg/kg b.w., about 120 mg/kg b.w., about 140 mg/kg b.w., about 160 mg/kg b.w., about 180 mg/kg b.w., about 200 mg/kg b.w., about 220 mg/kg b.w., about 240 mg/kg b.w., about 260 mg/kg b.w., about 280 mg/kg b.w., about 300 mg/kg b.w., about 320 mg/kg b.w., about 340 mg/kg b.w., about 360 mg/kg b.w., about 380 mg/kg b.w., about 400 mg/kg b.w., about 420 mg/kg b.w., about 440 mg/kg b.w., about 460 mg/kg b.w., about 480 mg/kg b.w., or about 500 mg/kg b.w. In one non-limiting embodiment of the invention, the composition is administered at about 150 mg/kg b.w. The composition can be administered one, two, three, four, five, six or more times per day, per week, per month, or combinations thereof. The administration schedule for the the composition can be modulated according to the patient's response to treatment.

The present disclosure is further described in the light of the following non-limiting examples which are set forth for illustration purposes only and are not to be construed as limiting the scope of the present invention.

### Examples

### Example 1—Making of an Embodiment of the Chlorogenic Acid Composition

100 kg of green coffee beans was stacked in a vertical 1.0 KL extractor. The bottom of the extractor comprised a perforated plate on which filtration cloth was fixed. About six bed volumes of 70% v/v ethyl alcohol was added. Extraction was continued at 75-78° C. for about 7-8 hrs with continuous circulation of extract with transfer pump. After completion of extraction, the extract was filtered through 5 micron SS candle filter and clear extract was collected in a cleaned receiver tank. The bed was re-extracted by adding 4 bed volumes of 70% ethyl alcohol three more times and the temperature was maintained at 75-78° C. for about 7-8 hrs. All the extracts were collected in a receiver tank and combined extract was concentrated in a reactor under vacuum at 80-5° C. until the extract was free from ethyl alcohol. A solution was made up to the TDS to 20-25 w/v % with de-ionized water.

The extract solution was passed through a 500 Liter of XAD-4 resin and the extract was loaded through the resin at the rate of 2-3 bed volumes/hour. The resin was washed with 2-3 bed volumes of de-ionized water at the rate of 2-3 bed volumes/hour. The extract was eluted with 2-3 bed volumes of 70 v/v % ethyl alcohol at the rate of 2-3 bed volumes/hour. The eluent was concentrated in a reactor at 80±5° C. until free from alcohol. The solution TDS was made up to 25-30 w/v % and spray dried at 215±5° C. to obtain the composition.

The composition of the chlorogenic acid isomers was determined by HPLC and the following results were obtained:

TABLE 1
Peak Table showing Types of Chlorogenic acids

| | | Ret. | | | | Height |
|---|---|---|---|---|---|---|
| Peak | Name | Time | Area | Area % | Height | % |
| 1 | 3 CQA | 3.209 | 1904533 | 12.529 | 395880 | 13.745 |
| 2 | 5 CQA | 5.017 | 7126683 | 46.883 | 1394953 | 48.431 |
| 3 | 4 CQA | 5.218 | 2612578 | 17.187 | 521622 | 18.110 |
| 4 | 5 FQA | 7.489 | 703664 | 4.629 | 136587 | 4.742 |
| 5 | 3,4-diCQA | 11.049 | 941037 | 6.191 | 162285 | 5.634 |
| 6 | 3,5 Di CQA | 11.549 | 694548 | 4.569 | 111103 | 3.857 |
| 7 | 4,5 Di CQA | 12.659 | 1218027 | 8.013 | 157843 | 5.480 |
| Total | | | 15201070 | 100 | 2880273 | 100 |

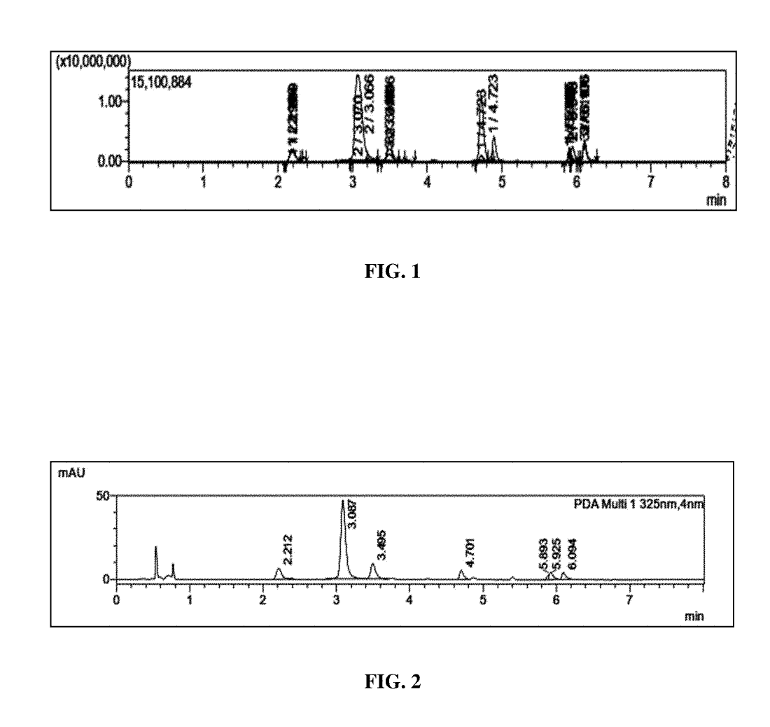

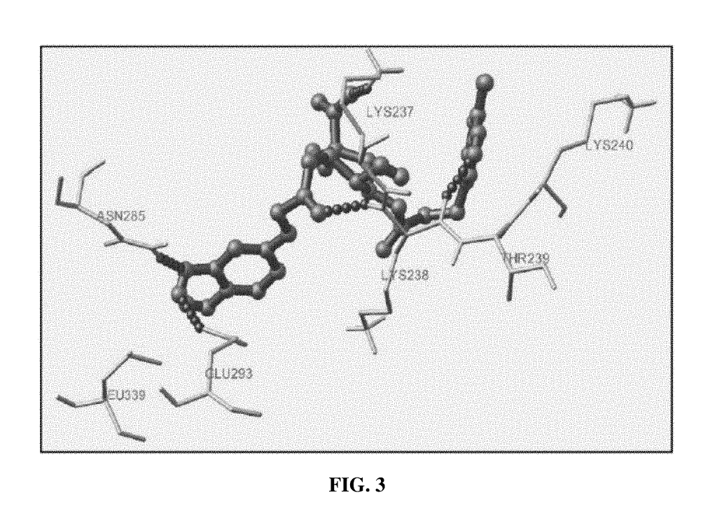

The chlorogenic acid isomer content was determined by LC-MS/MS (see FIGS. 1 and 2). The composition of this Example 1 was used in the following examples.

### Example 2—In-Vitro Studies

Antioxidant Assay

DPPH Scavenging Assay

The free radical scavenging capacity of the composition of Example 1 was determined using DPPH scavenging assay (Sarojini et al., 2011). DPPH solution was prepared in 95% methanol. Freshly prepared DPPH solution was taken in test tubes and the composition was added and incubated for 20 min. The absorbance was read at 515 nm using a spectrophotometer. A blank was prepared containing the same volume of reaction mixture without any tested samples. The percentage of scavenging was calculated using formula:

% Scavenging=Ac−As/Ac×100
Where Ac was the absorbance of the control (blank without extract) and As was the absorbance in the presence of the extract

Metal Chelating Activity

The chelating of ferrous ions by the composition was determined by the following method. The composition of Example 1 was added to a solution of 2 mM FeCl2 (0.05 ml), the reaction was initiated by the addition of 5 mM ferrozine (0.2 ml). Then the mixture was shaken vigorously and kept for 10 min at room temperature. Absorbance of the solution was measured at 562 nm. The percentage inhibition of ferrozine-Fe2+ complex formation was calculated as follows:

Chelating rate (%)=Ac−As/Ac×100
Where Ac was the absorbance of the control (blank without extract) and As was the absorbance in the presence of the extract

Superoxide Anion Scavenging Activity

Superoxide anion scavenging activity of the composition of Example 1 was measured according to the method of (Nishimiki et al., 1972). All the solutions in this experiment were prepared using phosphate buffer (pH 7.4). Add 1 ml of NBT (156 μM), 1 ml of NADH (468 μM) and 3 ml of test samples. The reaction was started by adding 100 ml of PMS (60 μM) and incubated at 25° C. for 5 min followed by measurement of absorbance at 560 nm. The percentage of scavenging was calculated using formula:

% Scavenging=Ac−As/Ac×100
Where Ac was the absorbance of the control (blank without extract) and As was the absorbance in the presence of the extract

Reducing Power Assay

The reductive ability of the composition of Example 1 was determined. The test samples were mixed with 2.5 ml of 0.2 M phosphate buffer (pH 6.6) and 2.5 ml of 1% potassium ferricyanide [K3Fe(CN)6]. The reaction mixture was incubated at 50° C. for 20 min, 2.5 ml of 10% trichloroacetic acid was added, then centrifuged (650 rpm at room temperature) for 10 min. The upper layer solution (2.5 ml) was mixed with 2.5 ml of distilled water and 0.5 ml of 0.1% FeCl3. Absorbance was measured at 700 nm. Higher absorbance at 700 nm indicated higher reducing power (Oyaizu, 1986).

Total Antioxidant Activity

The phosphomolybdenum method is based on the reduction of Mo (VI) to Mo (V) by the antioxidant compound and the formation of a green phosphate/Mo (V) complex with a maximal absorption at 695 nm. The antioxidant activity of the test sample was determined by the phosphomolybdenum method as described by Prieto et al., (1999). Briefly, 0.3 ml of test sample was combined with 3 ml of reagent solution (0.6M sulfuric acid, 28 mM sodium phosphate and 4 mM ammonium molybdate). The reaction mixture was incubated at 95° C. for 90 min and cooled to room temperature. The absorbance of the solution was measured at 695 nm against blank. The total antioxidant capacity was expressed as the number of equivalents of ascorbic acid (AAE).

Results

TABLE 2
In Vitro Results for the Chlorogenic Acid Composition

| | | | Superoxide scavenging | | | | | |
|---|---|---|---|---|---|---|---|---|
| DPPH scavenging assay | | | assay | | | Metal chelating | | |
| Conc | | | Conc | | | Conc | | |
| in | Absorbance | % | in | Absorbance | % | in | Absorbance | % |
| μg/ml | @517 nm | inhibition | μg/ml | @560 nm | inhibition | μg/ml | @562 nm | inhibition |
| Blank | 1.801 | | Blank | 0.337 | | Blank | 0.702 | |
| 100 μg | 0.886 | 50.81 | 100 μg | 0.110 | 67.36 | 100 μg | 0.726 | −3.42 |
| | 1.007 | 44.09 | | 0.102 | 69.73 | | 0.739 | −5.27 |
| | 0.985 | 45.31 | | 0.120 | 64.39 | | 0.746 | −6.27 |
| | 0.967 | 46.31 | | 0.123 | 63.50 | | 0.734 | −4.56 |
| | 0.976 | 45.81 | | | | | | |
| | | 45.97 | | | 66.25 | | | −4.88 |

| Total antioxidant assay | | | Reducing power assay | | |
|---|---|---|---|---|---|
| Conc in | Absorbance | | Conc in | Absorbance | |
| μg/ml | @695 nm | AAE (μg) | μg/ml | @700 nm | AAE (μg) |
| 100 μg | 0.078 | 14.78 | 100 μg | 0.545 | 58.55 |
| | 0.078 | 14.80 | | 0.590 | 63.37 |
| | 0.080 | 14.95 | | 0.598 | 64.16 |
| | 0.074 | 14.50 | | 0.560 | 60.09 |
| | 0.078 | 14.80 | | 0.555 | 59.63 |
| | | 14.76 | | | 61.16 |

### Example 3—Analgesic Activity

The analgesic property of the composition of Example 1 was tested using in vivo experiments. These studies included the peripheral and central analgesic activity of the composition using acetic acid induced writhing and Eddy's hot plate tests in mice. In both the models, it was evident that composition was effective in exerting anti-nociceptive activity in a dose-dependent manner and showed best results at 150 mg/kg b.w. (data not shown). To gain further insights into the molecular mechanism behind the analgesic activity of the composition, in silico analysis of molecular interactions between the isomers of the composition and TrpV1 was evaluated. The following study was carried out to see whether or not the active principles of the composition inhibit TrpV1 at its binding site.

### Example 4—Molecular Interaction of the Chlorogenic Acid Composition with the TrpV1 Calmodulin Binding Site

Molecular docking is an important tool for identifying enzyme-ligand interactions. In the present study, the chlorogenic acids in the composition of Example 1 were subjected to virtual screening for the ability to inhibit TrpV1 function.

Methods

The composition of Example 1 was subjected to binding affinity analysis for the TrpV1 binding site using DogSiteScorer (Volkamer et al. 2012). AutoDock tools were utilized to generate grids, calculate dock scores, and evaluate the conformers of chlorogenic acids in the composition bound to the active site of TrpV1 for analgesic activity. Automated docking is a graphical user interface. AutoDock 4.2 was employed to get docking and binding scores, which was implemented by the Lamarckian genetic algorithm method. The ligand molecules were designed and the structure was analyzed using ACD/Chemsketch. The PRODRG server was used to minimize energy of drug compounds and 3D coordinates were prepared. The protein structure file (PDB ID: 2PNN) was taken from PDB and edited by removing the hetero atoms using Python molecule viewer. The grid map was centered at particular residues of the protein and was generated with AutoGrid. As per genetic algorithm, all the torsions were allowed to rotate during docking. The Lamarckian genetic algorithm and the pseudo-Solis and Wets methods were applied for minimization, using default parameters (Rodriguez and Infante, 2011).

Result

TABLE 3
Size and Shape Descriptors of Active Pocket

| Descriptor | Value |
|---|---|
| Volume [Å³] | 444.03 |
| surface [Å²] | 766.79 |
| lipophilic surface [Å²] | 593.70 |
| depth [Å] | 17.48 |
| ellipsoid main axis ratio c/a | 0.12 |
| ellipsoid main axis ratio b/a | 0.47 |
| enclosure | 0.18 |

TABLE 4
Element Descriptors of Protein Active Site

| Descriptor | Value |
|---|---|
| Pocket atoms | 84 |
| Carbons | 61 |
| Nitrogen | 9 |
| Oxygen | 14 |
| Sulfur | 0 |
| Other elements | 0 |

TABLE 5
Amino Acid Composition

| Descriptor | Value |
|---|---|
| Apolar amino acid ratio | 0.33 |
| Polar amino acid ratio | 0.38 |
| Positive amino acid ratio | 0.14 |
| Negative amino acid ratio | 0.14 |

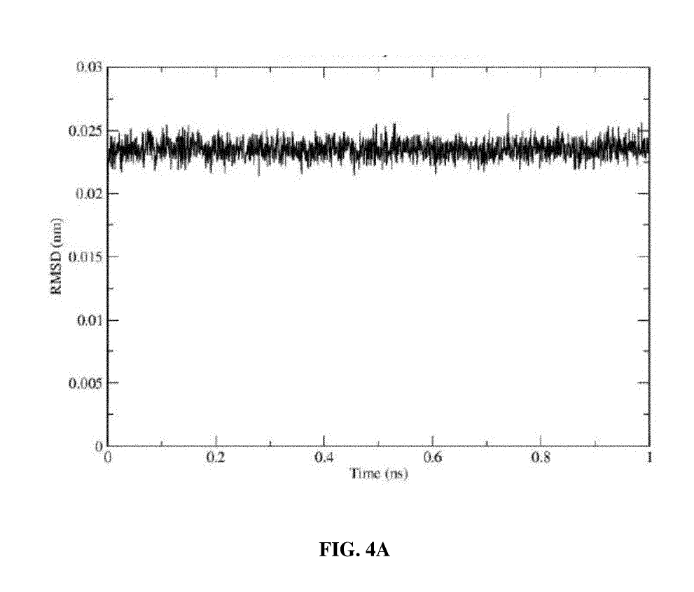

In the present study, the chlorogenic acids in the composition of Example 1 were analyzed for the inhibition of TrpV1 activity. The analysis showed that isomers of the composition effectively interacted with the calmodulin binding site residues of enzyme TrpV1 indicating the composition as a TrpV1 antagonist (FIG. 3). Among the isomers, 3,5-diCQA interacted with top binding conformation (binding energy of −5.46 KJ/mol) having 5 hydrogen bonds with the Lys238, Glu293, Asn285, Lys237 residues of TrpV1 active pocket (Table 6). After comparative docking it was observed that the isomers of the composition showed better interactions with the calmodulin-binding site of protein than the standard drug paracetamol.

TABLE 6
Molecular docking results of TrpV1 with active principles of the
composition

| | Binding | Ligand | Inhibitory | Intermolecular | | |
|---|---|---|---|---|---|---|
| | Energy | Efficiency | Constant | energy | | |
| Phytoconstituent | (KJ/mol) | (KJ/mol) | (μM) | KJ/mol | H-bonds | Interactions |
| 3-O- | −4.02 | −0.16 | 1.12 | −7.31 | 4 | Asn285, Lys332 |
| Caffeoylquinic | | | | | | Glu293 |
| acid | | | | | | |
| 4-O- | −4.64 | −0.19 | 395.63 | −7.92 | 3 | Ser282, Asn285 |
| Caffeoylquinic | | | | | | Lys237 |
| acid | | | | | | |
| 5-O- | −3.42 | −0.14 | 3.1 | −6.7 | 3 | Lys237, Lys332 |
| Caffeoylquinic | | | | | | Lys238 |
| acid | | | | | | |
| 5-O- | −5.86 | −0.23 | 50.82 | −9.14 | 4 | Ser342, Lys238, |
| Feruloylquinic | | | | | | Lys332 |
| acid | | | | | | |
| 3,4-O- | −2.29 | −0.06 | 20.98 | −7.36 | 3 | Val283, Lys332, |
| Dicaffeoylquinic | | | | | | Ser282 |
| acid | | | | | | |
| 3,5-O- | −6.46 | −0.15 | 99.97 | −10.63 | 6 | Lys238, Glu293, |
| Dicaffeoylquinic | | | | | | Asn286, Lys237 |
| acid | | | | | | |
| 4,5-O- | −4.53 | −0.20 | 33.58 | −6.5 | 3 | Lys237, Asn285, |
| Dicaffeoylquinic | | | | | | Lys238 |
| acid | | | | | | |
| Paracetamol | −4.11 | −0.37 | 973.71 | −4.71 | 2 | Glu293, Lys238 |

### Example 5—Molecular Dynamics of the Chlorogenic Acid Composition/TrpV1 Interaction

Molecular dynamic (MD) simulation is a powerful tool for studying the binding mechanism at the atomic level (Leszczynski, 2005). In this study, MD simulation was used to investigate the detailed interactions between TrpV1 calmodulin binding site and 3,5-diCQA.

Method

In order to study the binding of 3,5-diCQA with the TrpV1 calmodulin binding site, the 1 ns molecular dynamics simulations were performed using the open GROMACS 5.0 package for Linux. Before the simulations, the coordinate file and topology file were prepared (Liu et al. 2013) and the water box was constructed and filled with simple point charge water solution, which was then neutralized by sodium ions or chloride ions (Bernardes et al. 2013). The 500-step energy minimization of the system was performed using the steepest descending method. The NVT (constant number, volume and temperature) ensembles were used with temperature being maintained at 300 K. The cutoff radius of van der Waals interaction was 1.4 nm, and particle mesh Ewald algorithm was used for the electrostatic interaction. The Linear Constraint Solver algorithm was used for all of the bond restrictions.

Results

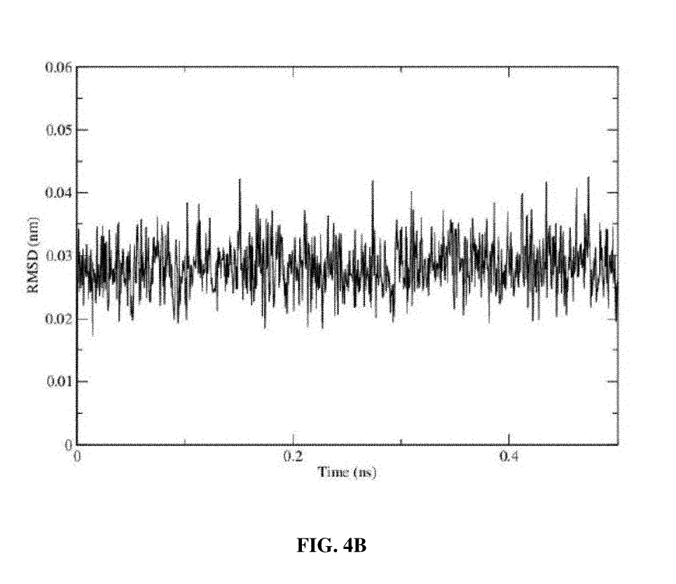

In order to study the dynamic behavior and the binding stability of the TrpV1 and 3,5-diCQA complex, the 1 ns molecular dynamics simulations were performed on protein-ligand complex. The simulation was performed for 1 nanosecond to ensure that the equilibration phase lasted long enough for further binding energy and hydrogen binding mode analysis. The RMSD versus the simulation time was considered as a significant criterion to evaluate the stability of dynamic behavior. The stability was evaluated by the RMSD values of the backbone atoms in the protein-ligand complex (FIG. 4A) and those of the molecule alone (FIG. 4B) relative to those in the starting structure of the heating phase. The trajectories for the simulation system remained stable throughout. It could easily be seen that the chlorogenic acid did not significantly fluctuate with time indicating firm and stable interaction with the protein TrpV1. The RMSD values were <0.05 nm (FIG. 4B).

Conclusions

The inventor's studies on experimental animals have successfully evaluated the central and peripheral analgesic properties of the chlorogenic acid composition of Example 1.

TrpV1 cation channels play a very crucial role in the modulation of nociceptive stimuli. Elevation in the levels of TrpV1 mediated Ca²⁺ ions in the cytosol are mainly responsible for the subsequent effects on downstream protein targets via calmodulin binding, thus mediating inflammatory pain.

The inventor found that the composition of Example 1 effectively binds and inhibits the calmodulin-binding site of TrpV1. The results were obtained by carrying out screening of molecules for the interaction with the TrpV1 active site.

Using molecular docking, it was evident that all the isomers of the composition of Example 1 can inhibit TrpV1 as compared to the standard drug paracetamol.

Molecular dynamic simulation studies were carried out in order to study the dynamics of the TrpV1/3,5-dicaffeoylquinic acid complex. The complex remained stable and the system equilibrated during the simulation time. This clearly indicates that the chlorogenic acid isomers interact firmly with TrpV1.

### Example 6—Interaction of the Composition with Trv1 and Trv4 Binding Sites

Transient receptor potential (TRP) channels represent a distinct group of ion channels that serve as cellular sensors for a broad spectrum of physical and chemical stimuli (Clapham, 2003; Ramsey et al. 2006). They are sensitive to activation by fundamental cell signaling elements such as Ca²⁺, PIP2, cyclic nucleotides, phosphorylation potential, temperature, and osmotic pressure, as well as environmental inputs that can be either beneficial or harmful. Upon activation, TRP channels change the membrane potential, translocate important signaling ions across the cell membrane, alter enzymatic activity, initiate endocytosis/exocytosis, and so on. There are about 28 Trp channels explored in mammalian species to date, classified as several subfamilies viz. TrpC (canonical), TrpV (vanilloid), TrpM (melastatin), TrpA (ankyrin), TrpML (mucolipin) and TrpPP (polycystin) (Clapham et al. 2001; Clapham, 2003).

TrpV1 and TrpV4 ion channels play a crucial role in the development of inflammatory hyperalgesia and pain (Basbaum et al. 2009; Dubin and Patapoutian, 2010). Studies have demonstrated changes in the expression of TRPV1 and TRPV4 associated with both mechanical and thermal hyperalgesia (Christoph et al. 2008; Ding et al. 2010). These channels are regulated in response to exogenous and endogenous vanilloids (pain evoking molecules) (Caterina and Julius, 2001; Van der Stelt and Di Marzo, 2004). In addition to vanilloids, the TrpV1 channel acts as a cellular sensor of acute noxious stimuli which includes heat, low pH and peptide toxins (Siemens et al. 2006; Bohlen et al. 2010). Several studies have been conducted to better understand the vanilloid mediated activation and deactivation of TrpV1, leading to the identification of vanilloid binding site (VBS) to which the vanilloids (capsaicin and Resiniferatoxin) bind, and evoke channel activation (Chou et al. 2004; Boukalova et al. 2010; Winter et al. 2013; Cao et al. 2013; Hanson et al. 2015).

In addition to the VBS, TrpV1 channel activation is regulated at a multiligand binding site for ATP and calmodulin present in the N-terminal ankyrin repeat domain (ARD) (Lishko et al. 2007). This ATP binding site is conserved in TrpV3 and TrpV4 as well (Phelps et al. 2010).

In the present investigation, it was shown that the amino acid residues of TrpV1 and TrpV4 interact with the isomers of the chlorogenic acid composition of Example 1 in the respective binding sites. An appreciable binding affinity was found for all the isomers of the chlorogenic acid composition with the regulatory sites of TrpV ion channels. This study provides new insights into the antinociceptive action of the chlorogenic acid composition through non-selective antagonism of Trp ion channels.

Method

Molecular dynamic simulation is a computational approach to study the interaction of ligands with target macromolecules. The three-dimensional crystal structures of TrpV1 in complex with capsazepine (PDB ID: 5IS0), ankyrin repeat domain of TrpV1 (PDB ID: 2PNN) and the human TrpV4 ankyrin repeat domain (PDB ID: 4DX2) were retrieved from the Protein Data Bank (http://www.rcsb.org/). AutoDock tool was utilized to generate grids, calculate dock score and evaluate the conformers of chlorogenic acid (CGA) isomers bound in the active sites of target proteins. AutoDock 4.2 was employed to get docking and binding scores; which was implemented by Lamarckian genetic algorithm method. The ligand molecules were designed and the structure was analyzed using ACD/Chemsketch. The PRODRG server was used to minimize energy of drug compounds and 3D coordinates were prepared. A grid box was generated using AutoGrid to define docking spaces for the respective proteins. The Lamarckian genetic algorithm and the pseudo-Solis and Wets methods were applied for minimization, using default parameters (Rodriguez and Infante, 2011). The lowest binding energy and the inhibitory constant for each protein were obtained from the docking log files (Dlg).

Results

Isomers of the Chlorogenic Acid Composition Interact Strongly with the Amino Acid Residues of the TrpV1 Vanilloid Binding Site

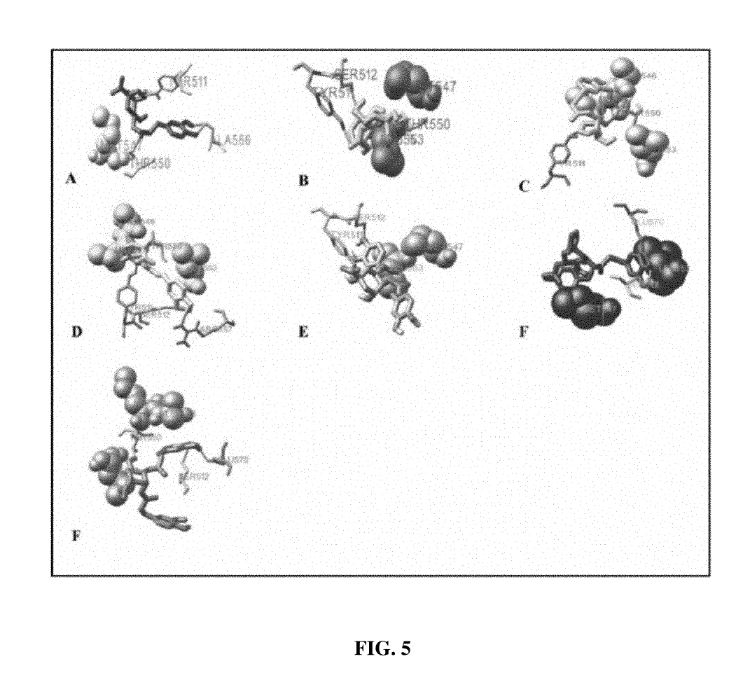

The present study performed docking analysis to understand the binding mode of the isomers of the chlorogenic acid composition into the VBS of TrpV1 (FIG. 5). All the seven isomers of the composition had a profound interaction with the key amino acid residues of the VBS. It was interesting to find that except for 3,5-Dicaffeoyl quinic acid (3,5-DiCQA) and 4,5-Dicaffeoyl quinic acid (4,5-DiCQA), all the isomers had common interaction with tyrosine at position 511. Further, the isomers such as 3-caffeoylquinic acid (3-CQA), 4-caffeoylquinic acid (4-CQA), 5-caffeoylquinic acid (5-CQA), 5-feruloylquinic acid (5-FQA) and 4,5-DiCQA were bound to the threonine 550 residue through hydrogen bonding. All the isomers had hydrophobic interaction with methionine at position 547. Other key interactions were serine 512, glutamic acid 570, alanine 566, arginine 557, leucine 553 and alanine 546 (Table 7).

In the present study, it was clearly demonstrated that the chlorogenic acid isomers had high binding affinity to key residues tyrosine 511 and serine 512. The tyrosine at position 511 was the first residue identified to participate in capsaicin and resiniferatoxin-evoked receptor activation (Jordt and Julius, 2002). Also, it was found that the isomers had profound interaction with threonine 550. Previous studies suggest that Thr550 is a crucial residue for competitive antagonist binding. Similar to capsazepine, the isomers had hydrophobic interactions with leucine 553 in the VBS. These results clearly show a high affinity binding of the chlorogenic acid isomers into the VBS of TrpV1.

TABLE 7
Molecular docking analysis of chlorogenic acid
isomers with the TrpV1 vanilloid binding site

| | Binding | Inhibitory | | | |
|---|---|---|---|---|---|
| | Energy | constant | | H-bond | Hydrophobic |
| Molecule | (kJ/mol) | (μM) | H-Bond | Interaction | interactions |
| 3-CQA | −7.36 | 4.03 | 5 | Tyr511, | Met547 |
| | | | | Thr550, | |
| | | | | Ala566 | |
| 4-CQA | −6.82 | 10.05 | 3 | Tyr511, | Met547, |
| | | | | Ser512, | Leu553 |
| | | | | Thr550 | |
| 5-CQA | −6.79 | 10.53 | 3 | Thr550, | Met547, |
| | | | | Tyr511 | Leu553, |
| | | | | | Ala546 |
| 5-FQA | −7.89 | 1.64 | 4 | Tyr511, | Met547, |
| | | | | Ser512, | Leu553, |
| | | | | Thr550, | Ala546 |
| | | | | Arg557 | |
| 3,4-DiCQA | −7.58 | 2.8 | 2 | Tyr511, | Met547, |
| | | | | Ser512 | Leu553 |
| 3,5-DiCQA | −7.23 | 5.03 | 3 | Ser512, | Met547, |
| | | | | Glu570 | Leu553 |
| 4,5-DiCQA | −7.54 | 2.98 | 3 | Ser512, | Met547, |
| | | | | Thr550, | Met552, |
| | | | | Glu570 | Leu553 |

3-CQA: 3-caffeoylquinic acid,
4-CQA: 4-caffeoylquinic acid,
5-CQA: 5-caffeoylquinic acid,
5-FQA: 5-caffeoylquinic acid,
3,4-DiCQA: 3,4-Dicaffeoylquinic acid,
3,5-DiCQA: 3,5-Dicaffeoylquinic acid,
4,5-DiCQA: 4,5-Dicaffeoylquinic acid

Isomers of the Composition Exhibit High Affinity Binding with the ATP-Calmodulin Binding Site of TrpV1

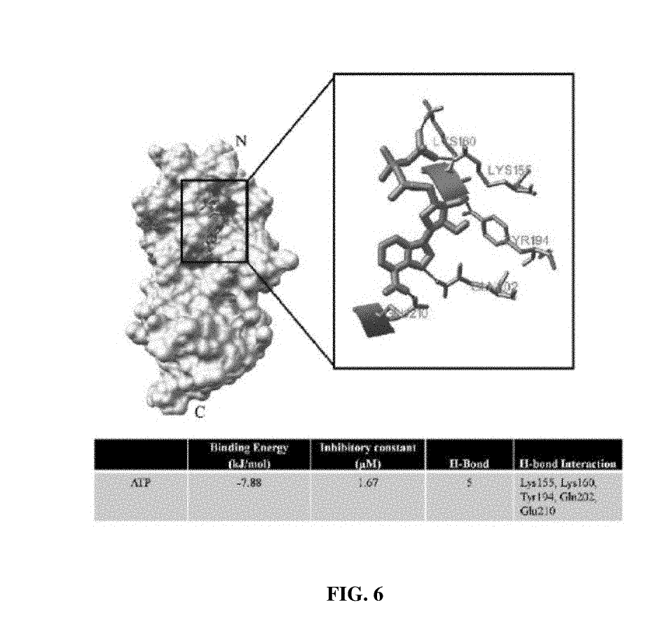

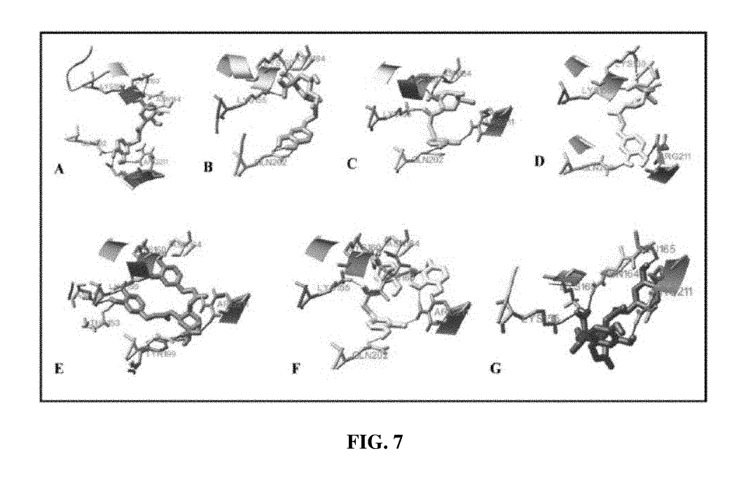

In the present study, a favorable interaction of the chlorogenic acid isomers with the multiligand binding site of TrpV1 was also shown. FIG. 6 shows the ankyrin repeat domain of TrpV1 bound to ATP. Similar to ATP, the isomers interacted with the binding site amino acids having profound H-bond interactions with key residues lys155 and lys 160. Further the isomers, except 4,5-DICQA, interacted commonly with glutamine at the 202 position. The lowest binding energy of all the isomers had a lower inhibitory constant indicating high affinity towards the ATP/CaM binding site of TrpV1 (Table 8). FIG. 7 shows the general area at which all the isomers can act upon the ATP-binding site.

The TrpV channels are regulated by the binding of ATP and Ca²⁺-CaM into a common site present in the N-terminal ARD. TrpV1 channel is sensitized by the binding of intracellular ATP, while it is inactivated by the Ca²⁺-CaM (Lishko et al. 2007). The isomers of chlorogenic acid in chlorogenic acid composition were shown to act as competitive antagonists of TrpV1 at its ARD. The interaction of the isomers with Lys155, Lysl60 and Glu202 provides striking evidence on the ability of the chlorogenic acid composition to modulate TrpV1 sensitivity.

TABLE 8
Docking score of chlorogenic acid composition
and ATP-CaM binding site of TrpV1

| | Binding | Inhibitory | | |
|---|---|---|---|---|
| | Energy | constant | | H-bond |
| Molecule | (kJ/mol) | (μM) | H-Bond | Interaction |
| 3-CQA | −6.16 | 30.47 | 6 | Lys160, Lys155, Gln202, |
| | | | | Asn164, Glu210, Arg211 |
| 4-CQA | −6.74 | 11.4 | 6 | Lys160, Lys155, Gln202, |
| | | | | Asn164 |
| 5-CQA | −6.22 | 27 63 | 5 | Lys160, Lys155, Asn164, |
| | | | | Gln202, Arg211 |
| 5-FQA | −8.13 | 1.09 | 5 | Lys160, Lys155, Asn164, |
| | | | | Gln202, Arg211 |
| 3,4-DiCQA | −6.3 | 23.94 | 9 | Lys160, Lys155, Thr153, |
| | | | | Asp150, Asn164, Tyr199, |
| | | | | Gln202, Arg211 |
| 3,5-DiCQA | −6.58 | 15.12 | 7 | Lys155, Lys160, Leu163, |
| | | | | Asn164, Gln202, Arg211 |
| 4,5-DiCQA | −7.02 | 7.19 | 5 | Lys155, Lys160, Asn164, |
| | | | | Leu163, Arg211 |

3-CQA: 3-caffeoylquinic acid,
4-CQA: 4-caffeoylquinic acid,
5-CQA: 5-caffeoylquinic acid,
5-FQA: 5-caffeoylquinic acid,
3,4-DiCQA: 3,4-Dicaffeoylquinic acid,
3,5-DiCQA: 3,5-Dicaffeoylquinic acid,
4,5-DiCQA: 4,5-Dicaffeoylquinic acid

Isomers of the Composition Exhibit Interaction with the ATP Binding Site of TrpV4

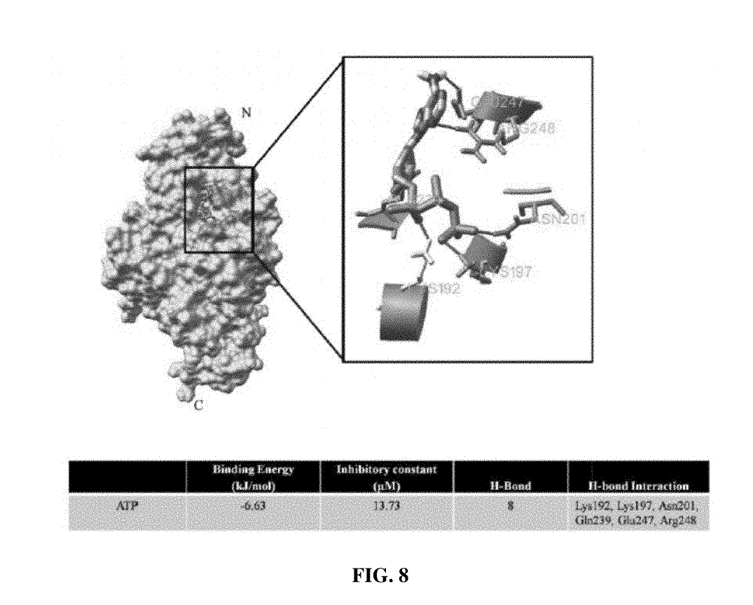

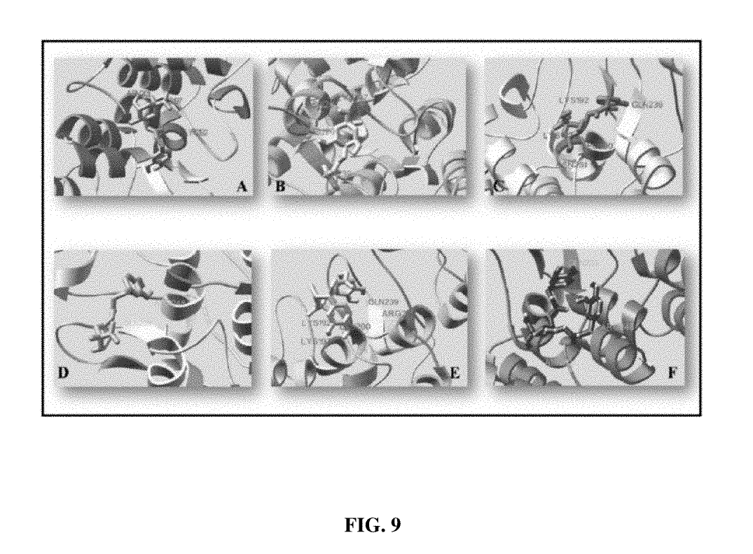

TrpV4 is another important member of Trp ion channels that take part in nociception (Shibasaki et al. 2007). Recently Lin et al. (2015) have demonstrated the overexpression of TrpV4 in the acid-induced fibromyalgia mouse model. In the present study, it was illustrated that the isomers of the chlorogenic acid composition exhibit high affinity binding into the ATP-CaM binding site of TrpV4. FIG. 8 shows the lowest energy binding pose of ATP into TrpV4-ARD. Amino acid residues Lys 192, Lys 197, Asn201, Gln239 and Arg248 are crucial determinants of ATP binding site of TrpV4. The interaction of ATP is similar to the rTrpV1-ARD (Lishko et al. 2007). Lys192 and Lys197 interact with the phosphate groups of ATP. These lysine residues are homologous to Lys155 and Lysl60 that are critical for ATP binding to TrpV1-ARD. Interestingly it was found that 3-CQA, 5-CQA and 3,5-DiCQA had H-bond interactions with Lys192 and Lys197 residues (FIG. 9). Further, the isomers were bound to Gln239 through H-bonding, indicating the similar mode of binding to ATP. Other interacting residues included Asn201, Arg248 and Leu200. The chlorogenic acid isomers were bound in the ATP binding site firmly as indicated by the hydrogen bonding with the neighboring residues and the low Ki values (Table 9). These indicate the isomers of the composition as competitive antagonists in the ATP binding pocket of TrpV4-ARD.

TABLE 9
Molecular docking score of interaction of CGA
isomers with ATP-CaM binding site of TrpV4 ARD

| | Binding | Inhibitory | | |
|---|---|---|---|---|
| | Energy | constant | | H-bond |
| Molecule | (kJ/mol) | (μM) | H-Bond | Interaction |
| 3-CQA | −5.96 | 42.68 | 4 | Lys192, Lys197, Asn201, |
| 4-CQA | −6.52 | 16.53 | 6 | Lys197, Leu200, Asn201, |
| | | | | Gln239 |
| 5-CQA | −6.43 | 19.25 | 7 | Lys192, Lys197, Leu200, |
| | | | | Asn201, Gln239 |
| 5-FQA | −7.9 | 1.61 | 3 | Gln239, Arg248 |
| 3,5-DiCQA | −6.36 | 21.83 | 6 | Lys192, Lys197, Leu200, |
| | | | | Gly239, Arg248 |
| 4,5-DiCQA | −5.94 | 44.05 | 6 | Asn201, Gln239, Arg248 |

3-CQA: 3-caffeoylquinic acid,
4-CQA: 4-caffeoylquinic acid,
5-CQA: 5-caffeoylquinic acid,
5-FQA: 5-caffeoylquinic acid,
3,4-DiCQA: 3,4-Dicaffeoylquinic acid,
3,5-DiCQA: 3,5-Dicaffeoylquinic acid,
4,5-DiCQA: 4,5-Dicaffeoylquinic acid

Conclusions

Trp ion channels, particularly the TrpV subfamily members, play a critical role in mechanical and thermal hyperalgesia. Targeting these ion channels for reducing the peripheral and central effects of nociception can provide next generation analgesics. The bioactive principles present in the chlorogenic acid composition are thus identified for the inactivation of TrpV channels. In this study, molecular docking tools were used to investigate the binding of the isomers of the chlorogenic acid composition into the respective binding sites of the TrpV1 and TrpV4 ion channels. The isomers showed favorable affinity to the specific binding sites of TrpV1 and TrpV4. The present findings show that the isomers of the chlorogenic acid composition can act as potent analgesics through non-selective antagonism of TrpV ion channels.

### REFERENCES

1. Anand P, Bley K. Topical capsaicin for pain management: Therapeutic potential and mechanisms of action of the new high-concentration capsaicin 8% patch. Br J Anaesth 2011; 107:490-502
2. Baradaran A, Madihi Y, Merrikhi A, Rafieian-Kopaei M, Nasri H. Serum lipoprotein in diabetic patients with various renal function not yet on dialysis. Pak J Med Sci 2013; 29(1): 354-357
3. Basbaum A I, Bautista D M, Scherrer G, Julius D. Cellular and molecular mechanisms of pain. Cell 2009; 139: 267-284
4. Bohlen C J, Priel A, Zhou S, King D, Siemens J, Julius D. A bivalent tarantula toxin activates the capsaicin receptor, TRPV1, by targeting the outer pore domain. Cell 2010; 141: 834-845
5. Bonica J J. The need of a taxonomy. Pain 1979; 6(3): 247-248
6. Boukalova S, Marsakova L, Teisinger J, Vlachova V. Conserved residues within the putative S4-S5 region serve distinct functions among thermosensitive vanilloid transient receptor potential (TRPV) channels. J. Biol. Chem. 2010; 285: 41455-41462
7. Cao E, Liao M, Cheng Y, Julius, D. TRPV1 structures in distinct conformations reveal activation mechanisms. Nature 2013; 504: 113-118
8. Caterina M J, Julius D. The vanilloid receptor: a molecular gateway to the pain pathway. Annu. Rev. Neurosci. 2001; 24: 487-517
9. Chou M Z, Mtui T, Gao Y-D, Kohler M, Middleton R E. Resiniferatoxin binds to the capsaicin receptor (TRPV1) near the extracellular side of the S4 transmembrane domain.

Biochemistry 2004; 43: 2501-2511
10. Christoph T, Bahrenberg G, De Vry J, Englberger W, Erdmann V A, Frech M, et al. Investigation of TRPV1 loss-of-function phenotypes in transgenic shRNA expressing and knockout mice. Mol Cell Neurosci 2008; 37: 579-589
11. Clapham D E, Runnels L W, Strubing C. The TRP ion channel family. Nat Rev Neurosci. 2001; 2: 387-396
12. Clapham D E. TRP channels as cellular sensors. Nature. 2003; 426: 517-524
13. Cui M, Honore P, Zhong C, Gauvin D, Mikusa J, Hernandez G et al. TrpV1 receptors in the CNS play a key role in broad-spectrum analgesia of TrpV1 antagonists. J Neurosci 2006; 26 (37): 9385-93
14. Ding X L, Wang Y H, Ning L P, Zhang Y, Ge H Y, Jiang H, et al. Involvement of TRPV4-NO-cGMP-PKG pathways in the development of thermal hyperalgesia following chronic compression of the dorsal root ganglion in rats. Behav Brain Res 2010; 208: 194-201
15. Dubin A E, Patapoutian A. Nociceptors: the sensors of the pain pathway. J. Clin. Invest. 2010; 120: 3760-3772
16. Everaerts W, Gees M, Alpizar Y A, Farre R, Leten C, Apetrei A et al. The capsaicin receptor TrpV1 is a crucial mediator of the noxious effects of mustard oil. Curr Biol 2011; 21(4): 316-21
17. Farshchi A, Ghiasi G, Abdollahuasl A. Antinociceptive and antiinflammatory effects of Teucrium hyrcanicum aqueous extract in male mice and rats. Physiol Pharmacol 2010; 14(1): 78-84
18. Gavva N R, Klionsky L, Qu Y, Shi L, Tamir R, Edenson S et al. Molecular determinants of vanilloid sensitivity in TRPV1. J Biol Chem. 2004; 279: 20283-20295
19. Hanson S M, Newstead S, Swartz K J, Sansom M S. Capsaicin interaction with TRPV1 channels in a lipid bilayer. molecular dynamics simulation. Biophys. J. 2015; 108: 1425-1434
20. Huang S M, Bisogno T, Trevisani M, Al-Hayani A, De Petrocellis L, Fezza F et al. An endogenous capsaicin-like substance with high potency at recombinant and native vanilloid VRI receptors. Proc Natl Acad Sci U.S.A. 2002; 99 (12): 8400-5
21. Jhaveri M D, Elmes S J, Kendall D A, Chapman V. Inhibition of peripheral vanilloid TrpV1 receptors reduces noxious heat-evoked responses of dorsal horn neurons in naïve, carrageenan-inflamed and neuropathic rats. Eur J Neurosci 2005; 22(2): 361-70
22. Jordt S E, Julius D. Molecular basis for species-specific sensitivity to “hot” chili peppers. Cell 2002; 108: 421-430
23. Leszczynski Jerzy. Computational chemistry: reviews of current trends, 2005; 9: 54-56
24. Lin J G, Hsieh C L, Lin Y W. Analgesic Effect of Electroacupuncture in a Mouse Fibromyalgia Model: Roles of TRPV1, TRPV4, and pERK. Plos One 2015; 10(6): e0128037
25. Lishko P V, Procko E, Jin X, Phelps C B, Gaudet R. Neuron 2007; 54: 905-918
26. Miladi Gorji H, Rashidi Pour A, Vafaei A A, Taherian A A. Opioid receptors role on anti-nociceptive effects of the aqueous extracts of Melissa officinalis in mice. J Hormozgan Univ Med Sci 2006; 10(1): 23-28
27. Nasri H, Shirzad H. Toxicity and safety of medicinal plants. J Herb Med Plarmacol 2013; 2(2): 21-22
28. Nolano M, Simone D A, Wendelschafer-Crabb G, Johnson T, Hazen E, Kennedy W R. Topical capsaicin in humans: Parallel loss of epidermal nerve fibers and pain sensation. Pain 1999; 81: 135-145
29. Phelps C B, Wang R R, Choo S S, Gaudet R. Differential Regulation of TRPV1, TRPV3, and TRPV4 Sensitivity through a Conserved Binding Site on the Ankyrin Repeat Domain. The Journal of Biological Chemistry. 2010; 285(1):731-740
30. Ramsey I S, Delling M, Clapham D E. An introduction to TRP channels. Annu Rev Physiol. 2006; 68: 619-647
31. Rodriguez A, Infante D. Characterization in silico of flavonoids biosynthesis in Theobroma cacao L. Net Biol 2011; 1: 34-45
32. Round P, Priestley A, Robinson J. An investigation of the safety and pharmacokinetics of the novel TRVP1 antagonist XEN-D0501 in healthy subjects. Br J Clin Pharmacol 2011; 72: 921-931
33. Rowbotham M C, Nothaft W, Duan W R, Wang Y, Faltynek C, McGaraughty S, Chu K L, Svensson P. Oral and cutaneous thermosensory profile of selective TrpV1 inhibition by ABT-102 in a randomized healthy volunteer trial. Pain 2011; 152: 1192-1200
34. Sewell R D E, Rafieian-Kopaei M. The history and ups and downs of herbal medicine usage. J HerbMed Pharmacol 2014; 3(1): 1-3
35. Shibasaki K, Suzuki M, Mizuno A, Tominaga M. Effects of body temperature on neural activity in the hippocampus: Regulation of resting membrane potentials by transient receptor potential vanilloid 4. J. Neurosci. 2007; 27: 1566-1575
36. Siemens J, Zhou S, Piskorowski R, Nikai T, Lumpkin E A, Basbaum A I et al. Spider toxins activate the capsaicin receptor to produce inflammatory pain. Nature 2006; 444: 208-212
37. Taherian A A, Rashidy-Pour A, Vafaei A A, Jarrahi M, MiladiGorgi, Emami-Abarghoii M et al. Assessment the effects of hydroalcoholic extract of Thymus vulgaris on acute pain in hot plate and tail flick in mice. Koomesh 2004; 5(3): 179-185
38. Van Der Stelt M, Di Marzo V. Endovanilloids: putative endogenous ligands of transient receptor potential vanilloid 1 channels. Eur. J. Biochem. 2004; 271: 1827-1834
39. Volkamer A, Kuhn D, Grombacher T, Rippmann F, Rarey M. Combining global and local measures for structure-based druggability predictions. J Chem Inf Model 2012; 52: 360-372
40. Winter Z, Buhala A, Otvos F, Jo\acute{ }svay K, Vizier C, Dombi G, Sza-konyi G, Ola\acute{ }h Z. Functionally important amino acid residues in the transient receptor potential vanilloid 1 (TRPV1) ion channel: an overview of the current mutational data. Mol. Pain 2013; 9:30

1. A method of treating pain comprising administering to a patient in need thereof a composition comprising 3-caffeoylquinic acid (3-CQA), 5-caffeoylquinic acid (5-CQA), 4-caffeoylquinic acid (4-CQA), 5-feruloylquinic acid (5-FQA), 3,4-dicaffeoylquinic acid (3,4-diCQA), 3,5-dicaffeoylquinic acid (3,5-diCQA), and 4,5-dicaffeoylquinic acid (4,5-diCQA).

2. The method of claim 1, wherein said composition comprises 3-CQA, 5-CQA, 4-CQA, 5-FQA, 3,4-diCQA, 3,5-diCQA, and 4,5-diCQA in a ratio of about 2.6:9.4:3.4:1.0:1.2:1.0:1.6.

3. The method of claim 1, wherein said composition comprises a mixture of chlorogenic acids consisting of about 13% 3-CQA, about 47% 5-CQA, about 17% 4-CQA, about 5% 5-FQA, about 6% 3,4-diCQA, about 5% 3,5-diCQA, and about 8% 4,5-diCQA.

4. The method of claim 1, wherein said composition comprises 3-CQA, 5-CQA, 4-CQA, 5-FQA, 3,4-diCQA, 3,5-diCQA, and 4,5-diCQA in a ratio of about 2.7:10.2:3.7:1.0:1.3:1.0:1.7.

5. The method of claim 1, wherein said composition comprises a mixture of chlorogenic acids consisting of 12.5% 3-CQA, 46.9% 5-CQA, 17.2% 4-CQA, 4.6% 5-FQA, 6.2% 3,4-diCQA, 4.6% 3,5-diCQA, and 8.0% 4,5-diCQA.

6. The method of claim 1, wherein said pain is selected from the group consisting of: acute pain; chronic pain; neuropathic pain; inflammatory pain; arthritis pain; migraine pain; headache pain; and combinations thereof.

7. The method of claim 1, wherein said composition has an antioxidant activity of about 46% at 100 μg/ml in vitro.

8. The method of claim 1, wherein said composition has a superoxide scavenging activity of about 66% at 100 μg/ml.

9. The method of claim 1, wherein said composition is an administration form selected from the group consisting of: a powder; liquid; pill; tablet; pellet; capsule; thin film; solution; spray; syrup; linctus; lozenge; pastille; chewing gum; paste; vapor; suspension; emulsion; ointment; cream; lotion; liniment; gel; drop; topical patch; buccal patch; bead; gummy; gel; sol; injection; and combinations thereof.

10. The method of claim 1, wherein said composition includes at least one of a vitamin, mineral, extract, amino acid, protein, carbohydrate, lipid, fatty acid, food, beverage, nutritional supplement, dietary supplement, excipient, pharmaceutically acceptable carrier, bulking agent, binding agent, caffeine, flavoring, sweetener, and preservative.

11. The method of claim 1, wherein said composition is administered systemically.

12. The method of claim 1, wherein said composition is administered by a route selected from the group consisting of: orally; buccally; sub-lingually; topically; parenterally; intravenously; intravaginally; rectally; by inhalation; and combinations thereof.

13. The method of claim 1, wherein said composition is administered orally.

14. The method of claim 1, wherein said composition is administered at a dosage of about 150 mg/kg body weight.

15. The method of claim 1, wherein said patient is human.

16. The method of claim 1, wherein said composition is a coffee bean extract.

17-28. (canceled)
